SEQUENCE LISTING (261 sequences)

SEQ ID NO: 1 - protein, 8 aa, synthetic construct
  REGION 1..8: note = nanobody sequence
GRTLRWPD

SEQ ID NO: 2 - protein, 8 aa, synthetic construct
  REGION 1..8: note = nanobody sequence
GRTLRWYD

SEQ ID NO: 3 - protein, 8 aa, synthetic construct
  REGION 1..8: note = nanobody sequence
GRTLRWCD

SEQ ID NO: 4 - protein, 8 aa, synthetic construct
  REGION 1..8: note = nanobody sequence
GFTFDGYD

SEQ ID NO: 5 - protein, 13 aa, synthetic construct
  REGION 1..13: note = nanobody sequence
IRTRPGRTFSSYD

SEQ ID NO: 6 - protein, 8 aa, synthetic construct
  REGION 1..8: note = nanobody sequence
GHTFARYN

SEQ ID NO: 7 - protein, 8 aa, synthetic construct
  REGION 1..8: note = nanobody sequence
GFAFSSYI

SEQ ID NO: 8 - protein, 5 aa, synthetic construct
  REGION 1..5: note = nanobody sequence
GRTGG

SEQ ID NO: 9 - protein, 8 aa, synthetic construct
  REGION 1..8: note = nanobody sequence
GSIFSFNS

SEQ ID NO: 10 - protein, 8 aa, synthetic construct
  REGION 1..8: note = nanobody sequence
GSIFSISA

SEQ ID NO: 11 - protein, 8 aa, synthetic construct
  REGION 1..8: note = nanobody sequence
GSIFSLNS

SEQ ID NO: 12 - protein, 8 aa, synthetic construct
  REGION 1..8: note = nanobody sequence
ESIRNINV

SEQ ID NO: 13 - protein, 9 aa, synthetic construct
  REGION 1..9: note = nanobody sequence
GGSIFSINA

SEQ ID NO: 14 - protein, 9 aa, synthetic construct
  REGION 1..9: note = nanobody sequence
SGSPFGITA

SEQ ID NO: 15 - protein, 8 aa, synthetic construct
  REGION 1..8: note = nanobody sequence
ERTGSNYA

SEQ ID NO: 16 - protein, 8 aa, synthetic construct
  REGION 1..8: note = nanobody sequence
GRTFSNYA

SEQ ID NO: 17 - protein, 8 aa, synthetic construct
  REGION 1..8: note = nanobody sequence
GRTFSNVA

SEQ ID NO: 18 - protein, 8 aa, synthetic construct
  REGION 1..8: note = nanobody sequence
GSTFSNVA

SEQ ID NO: 19 - protein, 8 aa, synthetic construct
  REGION 1..8: note = nanobody sequence
GFSFDDYA

SEQ ID NO: 20 - protein, 8 aa, synthetic construct
  REGION 1..8: note = nanobody sequence
GLSFDDYA

SEQ ID NO: 21 - protein, 8 aa, synthetic construct
  REGION 1..8: note = nanobody sequence
GFTFDDYA

SEQ ID NO: 22 - protein, 8 aa, synthetic construct
  REGION 1..8: note = nanobody sequence
GRTFGSHN

SEQ ID NO: 23 - protein, 8 aa, synthetic construct
  REGION 1..8: note = nanobody sequence
GPAVSSYT

SEQ ID NO: 24 - protein, 6 aa, synthetic construct
  REGION 1..6: note = nanobody sequence
GRTLYD

SEQ ID NO: 25 - protein, 6 aa, synthetic construct
  REGION 1..6: note = nanobody sequence
ARTLYD

SEQ ID NO: 26 - protein, 8 aa, synthetic construct
  REGION 1..8: note = nanobody sequence
GFTFEHYL

SEQ ID NO: 27 - protein, 8 aa, synthetic construct
  REGION 1..8: note = nanobody sequence
GFTFNNYF

SEQ ID NO: 28 - protein, 8 aa, synthetic construct
  REGION 1..8: note = nanobody sequence
GFTFSNYI

SEQ ID NO: 29 - protein, 8 aa, synthetic construct
  REGION 1..8: note = nanobody sequence
GSLLNYDA

SEQ ID NO: 30 - protein, 8 aa, synthetic construct
  REGION 1..8: note = nanobody sequence
GRTFSRIT

SEQ ID NO: 31 - protein, 8 aa, synthetic construct
  REGION 1..8: note = nanobody sequence
GFTFSNYY

SEQ ID NO: 32 - protein, 8 aa, synthetic construct
  REGION 1..8: note = nanobody sequence
GFAFSTYV

SEQ ID NO: 33 - protein, 8 aa, synthetic construct
  REGION 1..8: note = nanobody sequence
GRIFNIYA

SEQ ID NO: 34 - protein, 8 aa, synthetic construct
  REGION 1..8: note = nanobody sequence
GRTFSSYA

SEQ ID NO: 35 - protein, 8 aa, synthetic construct
  REGION 1..8: note = nanobody sequence
GVTFSPYA

SEQ ID NO: 36 - protein, 8 aa, synthetic construct
  REGION 1..8: note = nanobody sequence
GVTSSPYA

SEQ ID NO: 37 - protein, 8 aa, synthetic construct
  REGION 1..8: note = nanobody sequence
GRDFTTTS

SEQ ID NO: 38 - protein, 8 aa, synthetic construct
  REGION 1..8: note = nanobody sequence
GGTFTDHD

SEQ ID NO: 39 - protein, 8 aa, synthetic construct
  REGION 1..8: note = nanobody sequence
GRTVSGLA

SEQ ID NO: 40 - protein, 8 aa, synthetic construct
  REGION 1..8: note = nanobody sequence
GRTVSGSA

SEQ ID NO: 41 - protein, 8 aa, synthetic construct
  REGION 1..8: note = nanobody sequence
ALTFSVRP

SEQ ID NO: 42 - protein, 8 aa, synthetic construct
  REGION 1..8: note = nanobody sequence
GFTFSRDW

SEQ ID NO: 43 - protein, 8 aa, synthetic construct
  REGION 1..8: note = nanobody sequence
GFTFSSFA

SEQ ID NO: 44 - protein, 8 aa, synthetic construct
  REGION 1..8: note = nanobody sequence
GFTFSTYL

SEQ ID NO: 45 - protein, 8 aa, synthetic construct
  REGION 1..8: note = nanobody sequence
GRTGSIYG

SEQ ID NO: 46 - protein, 8 aa, synthetic construct
  REGION 1..8: note = nanobody sequence
GFPFSYTV

SEQ ID NO: 47 - protein, 8 aa, synthetic construct
  REGION 1..8: note = nanobody sequence
GFTFSRYP

SEQ ID NO: 48 - protein, 8 aa, synthetic construct
  REGION 1..8: note = nanobody sequence
GFVFSKNL

SEQ ID NO: 49 - protein, 8 aa, synthetic construct
  REGION 1..8: note = nanobody sequence
ARTFSIYT

SEQ ID NO: 50 - protein, 8 aa, synthetic construct
  REGION 1..8: note = nanobody sequence
KRIFSLYN

SEQ ID NO: 51 - protein, 8 aa, synthetic construct
  REGION 1..8: note = nanobody sequence
GFPLDYYA

SEQ ID NO: 52 - protein, 8 aa, synthetic construct
  REGION 1..8: note = nanobody sequence
GRTNSGDS

SEQ ID NO: 53 - protein, 8 aa, synthetic construct
  REGION 1..8: note = nanobody sequence
GFTISSYA

SEQ ID NO: 54 - protein, 8 aa, synthetic construct
  REGION 1..8: note = nanobody sequence
GFTFSSYY

SEQ ID NO: 55 - protein, 8 aa, synthetic construct
  REGION 1..8: note = nanobody sequence
GGTFSRYS

SEQ ID NO: 56 - protein, 8 aa, synthetic construct
  REGION 1..8: note = nanobody sequence
GSTFSSYA

SEQ ID NO: 57 - protein, 8 aa, synthetic construct
  REGION 1..8: note = nanobody sequence
GLTFSTYT

SEQ ID NO: 58 - protein, 8 aa, synthetic construct
  REGION 1..8: note = nanobody sequence
GRTFSTIG

SEQ ID NO: 59 - protein, 8 aa, synthetic construct
  REGION 1..8: note = nanobody sequence
ISGGREDT

SEQ ID NO: 60 - protein, 8 aa, synthetic construct
  REGION 1..8: note = nanobody sequence
ISGGRDST

SEQ ID NO: 61 - protein, 8 aa, synthetic construct
  REGION 1..8: note = nanobody sequence
ISGGPDST

SEQ ID NO: 62 - protein, 8 aa, synthetic construct
  REGION 1..8: note = nanobody sequence
ISPDGTKT

SEQ ID NO: 63 - protein, 8 aa, synthetic construct
  REGION 1..8: note = nanobody sequence
ISWSGRST

SEQ ID NO: 64 - protein, 7 aa, synthetic construct
  REGION 1..7: note = nanobody sequence
IISGGAT

SEQ ID NO: 65 - protein, 7 aa, synthetic construct
  REGION 1..7: note = nanobody sequence
IISGGST

SEQ ID NO: 66 - protein, 7 aa, synthetic construct
  REGION 1..7: note = nanobody sequence
ITSIGST

SEQ ID NO: 67 - protein, 7 aa, synthetic construct
  REGION 1..7: note = nanobody sequence
ITSGDST

SEQ ID NO: 68 - protein, 8 aa, synthetic construct
  REGION 1..8: note = nanobody sequence
ISWSGHST

SEQ ID NO: 69 - protein, 8 aa, synthetic construct
  REGION 1..8: note = nanobody sequence
ISSRDGST

SEQ ID NO: 70 - protein, 8 aa, synthetic construct
  REGION 1..8: note = nanobody sequence
ISSSDGST

SEQ ID NO: 71 - protein, 8 aa, synthetic construct
  REGION 1..8: note = nanobody sequence
IGSRDGST

SEQ ID NO: 72 - protein, 8 aa, synthetic construct
  REGION 1..8: note = nanobody sequence
ISWSGRTT

SEQ ID NO: 73 - protein, 10 aa, synthetic construct
  REGION 1..10: note = nanobody sequence
ISRDQTGDTT

SEQ ID NO: 74 - protein, 10 aa, synthetic construct
  REGION 1..10: note = nanobody sequence
ISRDQTGNTT

SEQ ID NO: 75 - protein, 8 aa, synthetic construct
  REGION 1..8: note = nanobody sequence
IEWSGIST

SEQ ID NO: 76 - protein, 7 aa, synthetic construct
  REGION 1..7: note = nanobody sequence
INWRGDT

SEQ ID NO: 77 - protein, 7 aa, synthetic construct
  REGION 1..7: note = nanobody sequence
INSRGDT

SEQ ID NO: 78 - protein, 8 aa, synthetic construct
  REGION 1..8: note = nanobody sequence
VYGPARST

SEQ ID NO: 79 - protein, 11 aa, synthetic construct
  REGION 1..11: note = nanobody sequence
INWGGRSGTNT

SEQ ID NO: 80 - protein, 8 aa, synthetic construct
  REGION 1..8: note = nanobody sequence
IESGGGGA

SEQ ID NO: 81 - protein, 8 aa, synthetic construct
  REGION 1..8: note = nanobody sequence
INPGGSNT

SEQ ID NO: 82 - protein, 7 aa, synthetic construct
  REGION 1..7: note = nanobody sequence
ISASGDT

SEQ ID NO: 83 - protein, 8 aa, synthetic construct
  REGION 1..8: note = nanobody sequence
ISWSGDIT

SEQ ID NO: 84 - protein, 7 aa, synthetic construct
  REGION 1..7: note = nanobody sequence
MHWYGGI

SEQ ID NO: 85 - protein, 7 aa, synthetic construct
  REGION 1..7: note = nanobody sequence
MHWYGGV

SEQ ID NO: 86 - protein, 8 aa, synthetic construct
  REGION 1..8: note = nanobody sequence
ISPNGGTT

SEQ ID NO: 87 - protein, 8 aa, synthetic construct
  REGION 1..8: note = nanobody sequence
ISWSGNTR

SEQ ID NO: 88 - protein, 8 aa, synthetic construct
  REGION 1..8: note = nanobody sequence
ISWNGAST

SEQ ID NO: 89 - protein, 7 aa, synthetic construct
  REGION 1..7: note = nanobody sequence
ITSSGST

SEQ ID NO: 90 - protein, 7 aa, synthetic construct
  REGION 1..7: note = nanobody sequence
IDSDGTT

SEQ ID NO: 91 - protein, 8 aa, synthetic construct
  REGION 1..8: note = nanobody sequence
IHSPSSHT

SEQ ID NO: 92 - protein, 8 aa, synthetic construct
  REGION 1..8: note = nanobody sequence
INDGGGSP

SEQ ID NO: 93 - protein, 8 aa, synthetic construct
  REGION 1..8: note = nanobody sequence
ISWIGTNT

SEQ ID NO: 94 - protein, 8 aa, synthetic construct
  REGION 1..8: note = nanobody sequence
ITHDSTTT

SEQ ID NO: 95 - protein, 8 aa, synthetic construct
  REGION 1..8: note = nanobody sequence
VDGHASKT

SEQ ID NO: 96 - protein, 7 aa, synthetic construct
  REGION 1..7: note = nanobody sequence
ISAHNTT

SEQ ID NO: 97 - protein, 8 aa, synthetic construct
  REGION 1..8: note = nanobody sequence
ISWSGGKT

SEQ ID NO: 98 - protein, 8 aa, synthetic construct
  REGION 1..8: note = nanobody sequence
ISVSGTTI

SEQ ID NO: 99 - protein, 8 aa, synthetic construct
  REGION 1..8: note = nanobody sequence
ISSSGGST

SEQ ID NO: 100 - protein, 8 aa, synthetic construct
  REGION 1..8: note = nanobody sequence
IRWSGTST

SEQ ID NO: 101 - protein, 8 aa, synthetic construct
  REGION 1..8: note = nanobody sequence
INSGGGST

SEQ ID NO: 102 - protein, 8 aa, synthetic construct
  REGION 1..8: note = nanobody sequence
SIPGGIRT

SEQ ID NO: 103 - protein, 8 aa, synthetic construct
  REGION 1..8: note = nanobody sequence
ISWTTGSA

SEQ ID NO: 104 - protein, 8 aa, synthetic construct
  REGION 1..8: note = nanobody sequence
ISWSGGTK

SEQ ID NO: 105 - protein, 8 aa, synthetic construct
  REGION 1..8: note = nanobody sequence
ISWSGTNP

SEQ ID NO: 106 - protein, 8 aa, synthetic construct
  REGION 1..8: note = nanobody sequence
ISWSAGST

SEQ ID NO: 107 - protein, 19 aa, synthetic construct
  REGION 1..19: note = nanobody sequence
AVRAVGTSYPSSKASLYNY

SEQ ID NO: 108 - protein, 19 aa, synthetic construct
  REGION 1..19: note = nanobody sequence
AVRAVGTTYPSSRASLYDY

SEQ ID NO: 109 - protein, 19 aa, synthetic construct
  REGION 1..19: note = nanobody sequence
AVRAVGTTYPSSRALLYDY

SEQ ID NO: 110 - protein, 7 aa, synthetic construct
  REGION 1..7: note = nanobody sequence
VKPGTPP

SEQ ID NO: 111 - protein, 19 aa, synthetic construct
  REGION 1..19: note = nanobody sequence
AAEKPGGGGSYYHTSTYDY

SEQ ID NO: 112 - protein, 18 aa, synthetic construct
  REGION 1..18: note = nanobody sequence
NAEGPPTYYSGAYSWDDY

SEQ ID NO: 113 - protein, 18 aa, synthetic construct
  REGION 1..18: note = nanobody sequence
NAEGPPVYYSGAYSWDDY

SEQ ID NO: 114 - protein, 18 aa, synthetic construct
  REGION 1..18: note = nanobody sequence
NAEGPPAYYSGAYSWDDY

SEQ ID NO: 115 - protein, 19 aa, synthetic construct
  REGION 1..19: note = nanobody sequence
AADYFDRGESYYFEATYDY

SEQ ID NO: 116 - protein, 19 aa, synthetic construct
  REGION 1..19: note = nanobody sequence
AADYFDRGGSYYFEATYDY

SEQ ID NO: 117 - protein, 20 aa, synthetic construct
  REGION 1..20: note = nanobody sequence
AAERSRGTAELCTFYYGMDY

SEQ ID NO: 118 - protein, 20 aa, synthetic construct
  REGION 1..20: note = nanobody sequence
AAERRRGNASPCTFYYGMDY

SEQ ID NO: 119 - protein, 19 aa, synthetic construct
  REGION 1..19: note = nanobody sequence
ARRGPAYSGSFHSIDTYDY

SEQ ID NO: 120 - protein, 19 aa, synthetic construct
  REGION 1..19: note = nanobody sequence
ATQSRPTSYPSSRQGSYDY

SEQ ID NO: 121 - protein, 19 aa, synthetic construct
  REGION 1..19: note = nanobody sequence
AAKDYGSVADYFKGYAYNY

SEQ ID NO: 122 - protein, 19 aa, synthetic construct
  REGION 1..19: note = nanobody sequence
AAKDYGSIADYFKGYAYNY

SEQ ID NO: 123 - protein, 19 aa, synthetic construct
  REGION 1..19: note = nanobody sequence
AAKDYGSLADYFKGYAYNY

SEQ ID NO: 124 - protein, 19 aa, synthetic construct
  REGION 1..19: note = nanobody sequence
AAKDYGSLADYFEGYAYKY

SEQ ID NO: 125 - protein, 10 aa, synthetic construct
  REGION 1..10: note = nanobody sequence
ARGYPGTRDL

SEQ ID NO: 126 - protein, 10 aa, synthetic construct
  REGION 1..10: note = nanobody sequence
ARGYPGTSDL

SEQ ID NO: 127 - protein, 13 aa, synthetic construct
  REGION 1..13: note = nanobody sequence
AADYRCYSTSPKY

SEQ ID NO: 128 - protein, 17 aa, synthetic construct
  REGION 1..17: note = nanobody sequence
AANGGLTIPTHHTSYDS

SEQ ID NO: 129 - protein, 9 aa, synthetic construct
  REGION 1..9: note = nanobody sequence
RTLISSRDA

SEQ ID NO: 130 - protein, 9 aa, synthetic construct
  REGION 1..9: note = nanobody sequence
RTLINSRDA

SEQ ID NO: 131 - protein, 9 aa, synthetic construct
  REGION 1..9: note = nanobody sequence
AQGLRVSST

SEQ ID NO: 132 - protein, 11 aa, synthetic construct
  REGION 1..11: note = nanobody sequence
AANQRGTGYQY

SEQ ID NO: 133 - protein, 12 aa, synthetic construct
  REGION 1..12: note = nanobody sequence
AAQPLIGQTYDY

SEQ ID NO: 134 - protein, 17 aa, synthetic construct
  REGION 1..17: note = nanobody sequence
AARSGSEGSSLRSEYDY

SEQ ID NO: 135 - protein, 17 aa, synthetic construct
  REGION 1..17: note = nanobody sequence
AARSGSEGSHLRYEYDY

SEQ ID NO: 136 - protein, 18 aa, synthetic construct
  REGION 1..18: note = nanobody sequence
AAGPYYAAAVTSLRAYTD

SEQ ID NO: 137 - protein, 19 aa, synthetic construct
  REGION 1..19: note = nanobody sequence
AARHPAAYGSYSDQSTYYA

SEQ ID NO: 138 - protein, 19 aa, synthetic construct
  REGION 1..19: note = nanobody sequence
AADRFSSTVVRRRPAEYDY

SEQ ID NO: 139 - protein, 5 aa, synthetic construct
  REGION 1..5: note = nanobody sequence
NDGPL

SEQ ID NO: 140 - protein, 6 aa, synthetic construct
  REGION 1..6: note = nanobody sequence
KMGGSG

SEQ ID NO: 141 - protein, 8 aa, synthetic construct
  REGION 1..8: note = nanobody sequence
TKEGRSDS

SEQ ID NO: 142 - protein, 9 aa, synthetic construct
  REGION 1..9: note = nanobody sequence
TTIFSGQMR

SEQ ID NO: 143 - protein, 10 aa, synthetic construct
  REGION 1..10: note = nanobody sequence
SAMVRGQDRY

SEQ ID NO: 144 - protein, 10 aa, synthetic construct
  REGION 1..10: note = nanobody sequence
AKGLNWRTAQ

SEQ ID NO: 145 - protein, 11 aa, synthetic construct
  REGION 1..11: note = nanobody sequence
VRGLDTGTAPQ

SEQ ID NO: 146 - protein, 11 aa, synthetic construct
  REGION 1..11: note = nanobody sequence
ARGGTNAIAER

SEQ ID NO: 147 - protein, 12 aa, synthetic construct
  REGION 1..12: note = nanobody sequence
GADLEGGGSYYG

SEQ ID NO: 148 - protein, 13 aa, synthetic construct
  REGION 1..13: note = nanobody sequence
AAKQNYGAGNYDY

SEQ ID NO: 149 - protein, 15 aa, synthetic construct
  REGION 1..15: note = nanobody sequence
ASMSSTWCVSWGADY

SEQ ID NO: 150 - protein, 16 aa, synthetic construct
  REGION 1..16: note = nanobody sequence
AATASLRSLTSRQYDY

SEQ ID NO: 151 - protein, 16 aa, synthetic construct
  REGION 1..16: note = nanobody sequence
QKLQYGYFPGSGAYSH

SEQ ID NO: 152 - protein, 16 aa, synthetic construct
  REGION 1..16: note = nanobody sequence
AADPRLLGSTSVSYKY

SEQ ID NO: 153 - protein, 17 aa, synthetic construct
  REGION 1..17: note = nanobody sequence
AAGLRRGSWYYTPDYDY

SEQ ID NO: 154 - protein, 18 aa, synthetic construct
  REGION 1..18: note = nanobody sequence
AADPVGGPGYDHINAYDY

SEQ ID NO: 155 - protein, 19 aa, synthetic construct
  REGION 1..19: note = nanobody sequence
AADRRLLYTLVYKANENTY

SEQ ID NO: 156 - protein, 19 aa, synthetic construct
  REGION 1..19: note = nanobody sequence
AGAEGIYYSGSCSYIESDY

SEQ ID NO: 157 - protein, 20 aa, synthetic construct
  REGION 1..20: note = nanobody sequence
VADVRNNGRESGLGSSSARY

SEQ ID NO: 158 - protein, 126 aa, synthetic construct
  REGION 1..126: note = nanobody sequence
QVQLQESGGGLVQAGASLRLSCVVAGRTLRWPDMGWFRQASGKEREFVAAISGGREDTNYSGSVKGRFTISRDNAKNTLYLQMNSLEPEDTAVYYCAVRAVGTSYPSSKASLYNYWGQGTQVTVSS

SEQ ID NO: 159 - protein, 126 aa, synthetic construct
  REGION 1..126: note = nanobody sequence
QVQLQESGGGLVQAGGSLRLSCAGSGRTLRWYDLGWFRQASGKEREFVAAISGGRDSTNYSGAVKGRFTISGDNAKNTVYLQMNSLEPEDTAVYYCAVRAVGTTYPSSRASLYDYWGQGTQVTVSS

SEQ ID NO: 160 - protein, 126 aa, synthetic construct
  REGION 1..126: note = nanobody sequence
QVQLQESGGGLVQAGGSLRLSCVVSGRTLRWCDMGWFRQVSGKEREFVAAISGGPDSTNYSGSVKGRFTISRDNAKNTVYLQMNSLEPEDAAVYYCAVRAVGTTYPSSRASLYDYWGQGTQVTVSS

SEQ ID NO: 161 - protein, 126 aa, synthetic construct
  REGION 1..126: note = nanobody sequence
QVQLQESGGGLVQAGGSLRLSCAAAGRTLRWYDLGWFRQASGKEREFVAAISGGRDSTNYSGAVKGRFTISGDNAKNTVYLQMNSLEPEDTAVYYCAVRAVGTTYPSSRASLYDYWGQGTQVTVSS

SEQ ID NO: 162 - protein, 126 aa, synthetic construct
  REGION 1..126: note = nanobody sequence
QVQLQESGGGLVQAGGSLRLSCAASGFTFDGYDIGWFRQASGKEREFVAAISGGRDSTNYSGAVKGRFTISGDNAKNTVYLQMNSLEPEDTAVYYCAVRAVGTTYPSSRALLYDYWGQGTQVTVSS

SEQ ID NO: 163 - protein, 126 aa, synthetic construct
  REGION 1..126: note = nanobody sequence
QVQLQESGGGLVQTGASLRLSCVVAGRTLRWYDLGWFRQASGKEREFVAAISGGRDSTNYSGAVKGRFTISGDNAKNTVYLQMNSLEPEDTAVYYCAVRAVGTTYPSSRASLYDYWGQGTQVTVSS

SEQ ID NO: 164 - protein, 131 aa, synthetic construct
  REGION 1..131: note = nanobody sequence
QVQLQESGGGSVQAGGSLRLSCAASIRTRPGRTFSSYDMGWFRQAPGKEREFVSAISGGRDSTNYSGAVKGRFTISGDNAKNTVYLQMNSLEPEDTAVYYCAVRAVGTTYPSSRASLYDYWGQGTQVTVSS

SEQ ID NO: 165 - protein, 126 aa, synthetic construct
  REGION 1..126: note = nanobody sequence
QVQLQESGGGLVQPGGSLRLSCAVSGHTFARYNMGWFRQAPGKEREFVAAISGGRDSTNYSGAVKGRFTISGDNAKNTVYLQMNSLEPEDTAVYYCAVRAVGTTYPSSRASLYDYWGQGTQVTVSS

SEQ ID NO: 166 - protein, 126 aa, synthetic construct
  REGION 1..126: note = nanobody sequence
QVQLQESGGGLVQAGGSLRLSCVVAGRTLRWYDLGWFRQASGKEREFVAAISGGRDSTNYSGAVKGRFTISGDNAKNTVYLQMNSLEPEDTAVYYCAVRAVGTTYPSSRASLYDYWGQGTQVTVSS

SEQ ID NO: 167 - protein, 126 aa, synthetic construct
  REGION 1..126: note = nanobody sequence
QVQLQESGGGLVQAGASLRLSCVVAGRTLRWYDLGWFRQASGKEREFVAAISGGRDSTNYSGAVKGRFTISGDNAKNTVYLQMNSLEPEDTAVYYCAVRAVGTTYPSSRASLYDYWGQGTQVTVSS

SEQ ID NO: 168 - protein, 126 aa, synthetic construct
  REGION 1..126: note = nanobody sequence
QVQLQESGGGLVQTGGSLRLSCVVAGRTLRWYDLGWFRQASGKEREFVAAISGGRDSTNYSGAVKGRFTISGDNAKNTVYLQMNSLEPEDTAVYYCAVRAVGTTYPSSRASLYDYWGQGTQVTVSS

SEQ ID NO: 169 - protein, 114 aa, synthetic construct
  REGION 1..114: note = nanobody sequence
QVQLQESGGGLVQPGGSLGLSCAASGFAFSSYIMNWVRQTPGKGLEWVSSISPDGTKTYYADSVKGRFTISRDNAKNTLHLQMNSLKSEDTAVYYCVKPGTPPPGQGTQVTVSS

SEQ ID NO: 170 - protein, 114 aa, synthetic construct
  REGION 1..114: note = nanobody sequence
QVQLQESGGGLVQAGGSLRLSCAASGFAFSSYIMNWVRQTPGKGLEWVSSISPDGTKTYYADSVKGRFTISRDNAKNTLHLQMNSLKSEDTAVYYCVKPGTPPPGQGTQVTVSS

SEQ ID NO: 171 - protein, 114 aa, synthetic construct
  REGION 1..114: note = nanobody sequence
QVQLQESGGGLVRPGGSLRLSCAASGFAFSSYIMNWVRQTPGKGLEWVSSISPDGTKTYYADSVKGRFTISRDNAKNTLHLQMNSLKSEDTAVYYCVKPGTPPPGQGTQVTVSS

SEQ ID NO: 172 - protein, 114 aa, synthetic construct
  REGION 1..114: note = nanobody sequence
QVQLQESGGGLVQPGGSLRLSCAASGFAFSSYIMNWVRQTPGKGLEWVSSISPDGTKTYYADSVKGRFTISRDNAKNTLHLQMSSLKSEDTAVYYCVKPGTPPSGQGTQVTVSS

SEQ ID NO: 173 - protein, 114 aa, synthetic construct
  REGION 1..114: note = nanobody sequence
QVQLQESGGGLVQTGGSLRLSCAASGFAFSSYIMNWVRQTPGKGLEWVSSISPDGTKTYYADSVKGRFTISRDNAKNTLHLQMNSLKSEDTAVYYCVKPGTPPPGQGTQVTVSS

SEQ ID NO: 174 - protein, 114 aa, synthetic construct
  REGION 1..114: note = nanobody sequence
QVQLQESGGGLVQPGGSLRLSCAASGFAFSSYIMNWVRQTPGKGLEWVSSISPDGTKTYYADSVKGRFTISRDNAKNTLHLQMNSLKSEDTAVYYCVKPGTPPPGQGTQVTVSS

SEQ ID NO: 175 - protein, 114 aa, synthetic construct
  REGION 1..114: note = nanobody sequence
QVQLQESGGGLAQPGGSLRLSCAASGFAFSSYIMNWVRQTPGKGLEWVSSISPDGTKTYYADSVKGRFTISRDNAKNTLHLQMNSLKSEDTAVYYCVKPGTPPPGQGTQVTVSS

SEQ ID NO: 176 - protein, 114 aa, synthetic construct
  REGION 1..114: note = nanobody sequence
QVQLQESGGGFVRPGGSLGLSCAASGFAFSSYIMNWVRQTPGKGLEWVSSISPDGTKTYYADSVKGRFTISRDNAKNTLHLQMNSLKSEDTAVYYCVKPGTPPPGQGTQVTVSS

SEQ ID NO: 177 - protein, 123 aa, synthetic construct
  REGION 1..123: note = nanobody sequence
QVQLQEAGGGLVQAGDSLRLSCLASGRTGGMGWFRQAPGKEREFVAAISWSGRSTYYTDSVKGRFTISRDDAKNTVYLQMNSLKPEDTAVYFCAAEKPGGGGSYYHTSTYDYWGQGTQVTVSS

SEQ ID NO: 178 - protein, 123 aa, synthetic construct
  REGION 1..123: note = nanobody sequence
QVQLQESGGGLVQTGDSLRLSCLASGRTGGMGWFRQAPGKEREFVAAISWSGRSTYYTDSVKGRFTISRDDAKNTVYLQMNSLKPEDTAVYFCAAEKPGGGGSYYHTSTYDYWGQGTQVTVSS

SEQ ID NO: 179 - protein, 123 aa, synthetic construct
  REGION 1..123: note = nanobody sequence
QVQLQESGGGLVQAGGSLRLSCLASGRTGGMGWFRQAPGKEREFVAAISWSGRSTYYTDSVKGRFTISRDDAKNTVYLQMNSLKPEDTAVYFCAAEKPGGGGSYYHTSTYDYWGQGTQVTVSS

SEQ ID NO: 180 - protein, 123 aa, synthetic construct
  REGION 1..123: note = nanobody sequence
QVQLQESGGGLVQAGDSLRLSCLASGRTGGMGWFRQAPGKEREFVAAISWSGRSTYYTDSVKGRFTISRDDAKNTVYLQMNSLKPEDTAVYFCAAEKPGGGGSYYHTSTYDYWGQGTQVTVSS

SEQ ID NO: 181 - protein, 123 aa, synthetic construct
  REGION 1..123: note = nanobody sequence
QVQLQESGGGLVQAGDSLRLSCLASGRTGGMGWFRQAPGKEREFVAAISWSGRSTYYTDSVKGRFTISRDDTKNTVYLQMNSLKPEDTAVYFCAAEKPGGGGSYYHTSTYDYWGQGTQVTVSS

SEQ ID NO: 182 - protein, 123 aa, synthetic construct
  REGION 1..123: note = nanobody sequence
QVQLQESGGGLAQAGDSLRLSCLASGRTGGMGWFRQAPGKEREFVAAISWSGRSTYYTDSVKGRFTISRDDAKNTVYLQMNSLKPEDTAVYFCAAEKPGGGGSYYHTSTYDYWGQGTQVTVSS

SEQ ID NO: 183 - protein, 123 aa, synthetic construct
  REGION 1..123: note = nanobody sequence
QVQLQESGGGLVQAGDSLRLSCLASGRTGGMGWFRQAPGKEREFVAAISWSGRSTYYTDSVKGRFTISRDDAKNTVYLQMNSLKPEDAAVYFCAAEKPGGGGSYYHTSTYDYWGQGTQVTVSS

SEQ ID NO: 184 - protein, 123 aa, synthetic construct
  REGION 1..123: note = nanobody sequence
QVQLQESGGGLVQAGDSLRLSCLASGRTGGMGWFRQAPGKEREFVAAISWSGRSTYYADSVKGRFTISRDDAKNTVHLQMNSLKPEDTAVYFCAAEKPGGGGSYYHTSTYDYWGQGTQVTVSS

SEQ ID NO: 185 - protein, 124 aa, synthetic construct
  REGION 1..124: note = nanobody sequence
QVQLQESGGGLVQAGGSLRLSCAASGSIFSFNSMAWYRQAPGKQRELVAAIISGGATNYADSVKGRFGISRDNAKNTVYLQMNSLKPEDTAVYFCNAEGPPTYYSGAYSWDDYWGQGTQVTVSS

SEQ ID NO: 186 - protein, 124 aa, synthetic construct
  REGION 1..124: note = nanobody sequence
QVQLQESGGGLVQPGGSLRLSCAASGSIFSISAMGWYRQAPGKQRELVAAIISGGSTTYAGSVKGRFSISRDDAKNTVYLQMNSLKPEDTAVYYCNAEGPPVYYSGAYSWDDYWGQGTQVTVSS

SEQ ID NO: 187 - protein, 124 aa, synthetic construct
  REGION 1..124: note = nanobody sequence
QVQLQESGGGLVQAGGSLRLSCAASGSIFSLNSMAWYRQAPGKQRELVAAIISGGSTTYAGSVKGRFSISRDDAKNTVYLQMNSLKPEDTAVYYCNAEGPPVYYSGAYSWDDYWGQGTQVTVSS

SEQ ID NO: 188 - protein, 124 aa, synthetic construct
  REGION 1..124: note = nanobody sequence
QVQLQESGGGLVQPGGSLRLSCTVSESIRNINVMGWYRQAPGKQRELVAAIISGGSTTYAGSVKGRFSISRDDAKNTVYLQMNSLKPEDTAVYYCNAEGPPVYYSGAYSWDDYWGQGTQVTVSP

SEQ ID NO: 189 - protein, 124 aa, synthetic construct
  REGION 1..124: note = nanobody sequence
QVQLQESGGGLVQAGGSLRLSCAASGSIFSLNSMAWYRQAPGKQRELVAAIISGGSTNYAGSVKGRFSISRDNAKNTVYLQMNSLKPEDTAVYYCNAEGPPAYYSGAYSWDDYWGQGTQVTVSS

SEQ ID NO: 190 - protein, 125 aa, synthetic construct
  REGION 1..125: note = nanobody sequence
QVQLQESGGGLVQPGGSLRLSCAASGGSIFSINAMGWYRQAPGNQRELVAAITSIGSTNYADSVKGRFTISRDKAKNTVYLQMNSLKPEDTAVYYCNAEGPPAYYSGAYSWDDYWGQGTQVTVSS

SEQ ID NO: 191 - protein, 125 aa, synthetic construct
  REGION 1..125: note = nanobody sequence
QVQLQESGGGLVQPGGSLRLSCTAASGSPFGITAMGWYRQAPGKQRELVAAITSGDSTNYADSVKGRFTISMDGAKNTVFLQMNSLKPEDTAVYYCNAEGPPVYYSGAYSWDDYWGQGTQVTVSS

SEQ ID NO: 192 - protein, 124 aa, synthetic construct
  REGION 1..124: note = nanobody sequence
QVQLQESGGGLVQTGGSLRLSCAASGSIFSLNSMAWYRQAPGKQRELVAAIISGGSTTYAGSVKGRFSISRDDAKNTVYLQMNSLKPEDTAVYYCNAEGPPVYYSGAYSWDDYWGQGTQVTVSS

SEQ ID NO: 193 - protein, 126 aa, synthetic construct
  REGION 1..126: note = nanobody sequence
QVQLQESGGGLVQAGDSLRLSCAASERTGSNYAMGWFRQAPGKEREFVAAISWSGHSTYYADSVEGRFTISRDDAKNTVYLQMNSLKPEDTAVYYCAADYFDRGESYYFEATYDYWGQGTQVTVSS

SEQ ID NO: 194 - protein, 126 aa, synthetic construct
  REGION 1..126: note = nanobody sequence
QVQLQESGGGLVQPGGSLRLSCAASGRTFSNYAMGWFRQAPGNEREFVAAISWSGHSTYYADSVEGRFTISRDDAKNTVYLQMNSLKPEDTAVYYCAADYFDRGESYYFEATYDYWGQGTQVTVSS

SEQ ID NO: 195 - protein, 126 aa, synthetic construct
  REGION 1..126: note = nanobody sequence
QVQLQESGGGLVQAGGSLRLSCAASGRTFSNVAMGWFRQAPGKGREFVAAISWSGRSTYYADSVEGRFTISRDDAKNTVYLQMNSLKPEDTAVYYCAADYFDRGGSYYFEATYDYWGQGTQVTVSS

SEQ ID NO: 196 - protein, 126 aa, synthetic construct
  REGION 1..126: note = nanobody sequence
QVQLQESGGGLVQAGDSLRLSCAASGRTFSNVAMGWFRQAPGKGREFVAAISWSGRSTYYADSVEGRFTISRDDAKNTVYLQMNSLKPEDTAVYYCAADYFDRGGSYYFEATYDYWGQGTQVTVSS

SEQ ID NO: 197 - protein, 126 aa, synthetic construct
  REGION 1..126: note = nanobody sequence
QVQLQESGGGLVQPGGSLRLSCAASGSTFSNVAMGWFRQAPGKGREFVAAISWSGRSTYYADSVEGRFTISRDDAKNTVYLQMNSLKPEDTAVYYCAADYFDRGGSYYFEATYDYWGQGTQVTVSS

SEQ ID NO: 198 - protein, 127 aa, synthetic construct
  REGION 1..127: note = nanobody sequence
QVQLQESGGGLVQAGGSLRLSCAASGFSFDDYAIGWFRQAPGKEREGVSCISSRDGSTSYADSVKGRFTVASDNAKNTVYLQMNKLKPEDTAVYYCAAERSRGTAELCTFYYGMDYWGKGTQVTVSS

SEQ ID NO: 199 - protein, 131 aa, synthetic construct
  REGION 1..131: note = nanobody sequence
QVQLQESGGGLVQTGGSLRLSCAVSGLSFDDYAVGWFRQAPGKEREGVSCISSSDGSTVYADSVKGRFTISNDNAKNNAKNMVYLQMNSLKPEDTAVYYCAAERRRGNASPCTFYYGMDYWGKGTQVTVSS

SEQ ID NO: 200 - protein, 127 aa, synthetic construct
  REGION 1..127: note = nanobody sequence
QVQLQESGGGLVQAGGSLRLSCEASGFTFDDYAIGWFRQAPGKEREGVSCIGSRDGSTSYADSVKGRFTVASDNAKNTVYLQMNKLKPEDTAVYYCAAERSRGTAELCTFYYGMDYWGKGTQVTVSS

SEQ ID NO: 201 - protein, 131 aa, synthetic construct
  REGION 1..131: note = nanobody sequence
QVQLQESGGGRVQPGGSLRLSCAASGFTFDDYAVGWFRQAPGKEREGVSCISSSDGSTVYADSVKGRFTISNDNAKNNAKNMVYLQMNSLKPEDTAVYYCAAERRRGNASPCTFYYGMDYWGKGTQVTVSS

SEQ ID NO: 202 - protein, 131 aa, synthetic construct
  REGION 1..131: note = nanobody sequence
QVQLQESGGGLVQAGGSLRLSCAASGFTFDDYAVGWFRQAPGKERVGVSCISSSDGSTVYADSVKGRFTISNDNAKNNAKNMVYLQMNSLKPEDTAVYYCAAERRRGNASPCTFYYGMDYWGKGTQVTVSS

SEQ ID NO: 203 - protein, 126 aa, synthetic construct
  REGION 1..126: note = nanobody sequence
QVQLQESGGGLVQAGGSLRLSCAASGRTFGSHNMGWFRQAPGQEREFVAAISWSGRTTYYADFVKGRLTISRDNAKNTLYLQMNSLKPEDTAVYYCARRGPAYSGSFHSIDTYDYWGQGTQVTVSS

SEQ ID NO: 204 - protein, 126 aa, synthetic construct
  REGION 1..126: note = nanobody sequence
QVQLQESGGGLVQAGGSLRLSCAASGRTFGSHNMGWFRQAPGQEREFVAAISWSGRTTYYADFVKGRFTISRDNAKNTLYLQMNSLKPEDTAVYYCARRGPAYSGSFHSIDTYDYWGQGTQVTVSS

SEQ ID NO: 205 - protein, 126 aa, synthetic construct
  REGION 1..126: note = nanobody sequence
  VARIANT 6: note = X can be any naturally occurring amino acid
QVQLQXSGGGLVQAGGSLRLSCAASGRTFGSHNMGWFRQAPGQEREFVAAISWSGRTTYYADFVKGRFTISRDNAKNTLYLQMNSLKPGDTAVYYCARRGPAYSGSFHSIDTYDYWGQGTQVTVSS

SEQ ID NO: 206 - protein, 126 aa, synthetic construct
  REGION 1..126: note = nanobody sequence
QVQLQESGGGLVQPGGSLRLSCAASGRTFGSHNMGWFRQAPGQEREFVAAISWSGRTTYYADFVKGRFTISRDNAKNTLYLQMNSLKPEDTAVYYCARRGPAYSGSFHSIDTYDYWGQGTQVTVSS

SEQ ID NO: 207 - protein, 128 aa, synthetic construct
  REGION 1..128: note = nanobody sequence
QVQLQESGGGLVQPGGSLRLSCAASGPAVSSYTTAWFRQASGKEREFVAGISRDQTGDTTNYSGSVKHRFTCSRDNAKNTVYLQMNNLTPEDTAVYFCATQSRPTSYPSSRQGSYDYWGQGTQVTVSS

SEQ ID NO: 208 - protein, 128 aa, synthetic construct
  REGION 1..128: note = nanobody sequence
QVQLQESGGGLVHPGGSLRLSCAASGPAVSSYTTAWFRQASGKEREFVAGISRDQTGDTTNYSGSVKHRFTCSRDNAKNTVYLQMNNLTPEDTAVYFCATQSRPTSYPSSRQGSYDYWGQGTQVTVSS

SEQ ID NO: 209 - protein, 128 aa, synthetic construct
  REGION 1..128: note = nanobody sequence
QVQLQESGGGLVQAGGSLRLSCAASGPAVSSYTTAWFRQASGKEREFVAGISRDQTGDTTNYSGSVKHRFTCSRDNAKNTVYLQMNNLTPEDTAVYFCATQSRPTSYPSSRQGSYDYWGQGTQVTVSS

SEQ ID NO: 210 - protein, 128 aa, synthetic construct
  REGION 1..128: note = nanobody sequence
QVQLQESGGGLVQPGGSLRLSCTASGPAVSSYTTAWFRQASGKEREFVAGISRDQTGNTTNYSGSVKHRFTCSRDNAKNTVYLQMNNLTPEDTAVYFCATQSRPTSYPSSRQGSYDYWGQGTQVTVSS

SEQ ID NO: 211 - protein, 122 aa, synthetic construct
  REGION 1..122: note = nanobody sequence
QVQLQESGGGLVQAGGSLRLSCAASGRTLYDMGWFRQAPGKEREFVAAIEWSGISTYYADSVKGRFTISRDGGFVYLQMNSLKPEDTAVYYCAAKDYGSVADYFKGYAYNYWGQGTQVTVSS

SEQ ID NO: 212 - protein, 122 aa, synthetic construct
  REGION 1..122: note = nanobody sequence
QVQLQESGGGLVQAGGSLRLSCAASARTLYDMGWFRQAPGKEREFVAAIEWSGISTYYADSVKGRFTISRDNGMVYLQMNSLKPEDTAVYYCAAKDYGSIADYFKGYAYNYWGQGTQVTVSP

SEQ ID NO: 213 - protein, 122 aa, synthetic construct
  REGION 1..122: note = nanobody sequence
QVQLQESGGGLVQTGGSLRLSCAASARTLYDMGWFRQAPGKEREFVAAIEWSGISTYYADSVKGRFTISRDNGMVYLQMTSLKPDDTAVYYCAAKDYGSLADYFKGYAYNYWGQGTQVTVSS

SEQ ID NO: 214 - protein, 124 aa, synthetic construct
  REGION 1..124: note = nanobody sequence
QVQLQESGGGLVQAGGSLRLSCAASGRTLYDMGWFRQAPGKEREFVAAIEWSGISTYYADSMKGRFTISRDDGGSMVYLQMISLKPEDTAVYYCAAKDYGSLADYFEGYAYKYWGRGTQVTVSS

SEQ ID NO: 215 - protein, 116 aa, synthetic construct
  REGION 1..116: note = nanobody sequence
QVQLQESGGGLVQPGGSLRLSCAASGFTFEHYLMSWVRQPPGKGPEWVSSINWRGDTYYTDSVKGRFTISRDNAKNTLYLQMNSLKPEDTALYYCARGYPGTRDLQGQGTQVTVSS

SEQ ID NO: 216 - protein, 116 aa, synthetic construct
  REGION 1..116: note = nanobody sequence
QVQLQGSGGGLVQPGGSLRLSCVASGFTFNNYFMSWVRQPPGKGPEWVSSINSRGDTYYTDSVKGRFTISRDNAKNTLYLQMNSLKPEDTALYYCARGYPGTSDLQGQGTQVTVSS

SEQ ID NO: 217 - protein, 116 aa, synthetic construct
  REGION 1..116: note = nanobody sequence
QVQLQESGGGLVQPGGSLRLSCVASGFTFSNYIMTWVRQPPGKGPEWVSSINWRGDTYYTDSVKGRFTISRDNAKNTLYLQMNSLKPEDTALYYCARGYPGTSDLQGQGTQVTVSS

SEQ ID NO: 218 - protein, 120 aa, synthetic construct
  REGION 1..120: note = nanobody sequence
QVQLQESGGGLVQAGDSLRLSCAVSGSLLNYDAITWFRQAPGKEHEGVSCVYGPARSTMYADSVQGRFVISRDNAKNTVSLQMNSLKPEDTAVYYCAADYRCYSTSPKYRGRGTQVTVSS

SEQ ID NO: 219 - protein, 120 aa, synthetic construct
  REGION 1..120: note = nanobody sequence
QVQLQESGGGLVRPGGSLRLSCAVSGSLLNYDAITWFRQAPGKEHEGVSCVYGPARSTMYADSVQGRFIISRDNAKNTVSLQMNSLKPEDTAVYYCAADYRCYSTSPKYRGRGTQVTVSS

SEQ ID NO: 220 - protein, 120 aa, synthetic construct
  REGION 1..120: note = nanobody sequence
QVQLQESGGGLVQPGGSLRLSCAVSGSLLNYDAITWFRQAPGKEHEGVSCVYGPARSTMYADSVQGRFIISRDNAKNTVSLQMNSLKPEDTAVYYCAADYRCYSTSPKYRGRGTQVTVSS

SEQ ID NO: 221 - protein, 127 aa, synthetic construct
  REGION 1..127: note = nanobody sequence
QVQLQESGGGLVQAGDSLRLSCAASGRTFSRITMGWFRQAPGKEREFVAAINWGGRSGTNTYYADSVKGRFAISKDNAKNTVYLQMNSLKPEDTAVYYCAANGGLTIPTHHTSYDSWGQGTQVTVSS

SEQ ID NO: 222 - protein, 127 aa, synthetic construct
  REGION 1..127: note = nanobody sequence
QVQLQESGGGLVQAGGSLRLSCAASGRTFSRITMGWFRQAPGKEREFVAAINWGGRSGTNTYYADSVKGRFAISKDNAKNTVYLQMNSLKPEDTAVYYCAANGGLTIPTHHTSYDSWGQGTQVTVSS

SEQ ID NO: 223 - protein, 127 aa, synthetic construct
  REGION 1..127: note = nanobody sequence
QVQLQESGGGLVQPGGSLRLSCAASGRTFSRITMGWFRQAPGKEREFVAAINWGGRSGTNTYYADSVKGRFAISKDNAKNTVYLQMNSLKPEDTAVYYCAANGGLTIPTHHTSYDSWGQGTQVTVSS

SEQ ID NO: 224 - protein, 116 aa, synthetic construct
  REGION 1..116: note = nanobody sequence
QVQLQESGGGLVQPGGSLRLSCAASGFTFSNYYMSWVRQAPGMGREWVSSIESGGGGATYADSVKGRFTISRDNAKNTLYLQMNSLKPEDTAVYYCRTLISSRDARGQGTQVTVSS

SEQ ID NO: 225 - protein, 116 aa, synthetic construct
  REGION 1..116: note = nanobody sequence
QVQLQESGGGHVQPGGSLRLSCAASGFTFSNYYMSWVRQAPGMGREWVSSIESGGGGATYADSVKGRFTISRDNAKNTLYLQMNSLKPEDTAVYYCRTLINSRDARGQGTQVTVSS

SEQ ID NO: 226 - protein, 116 aa, synthetic construct
  REGION 1..116: note = nanobody sequence
QVQLQESGGGLVQAGGSLRLSCAASGFTFSNYYMSWVRQAPGMGREWVSSIESGGGGATYADSVKGRFTISRDNAKNTLYLQMNSLKPEDTAVYYCRTLINSRDARGQGTQVTVSS

SEQ ID NO: 227 - protein, 116 aa, synthetic construct
  REGION 1..116: note = nanobody sequence
QVQLQESGGGLVQPGGSLRLSCAASGFAFSTYVMTWVRQAPGKGLEWVSSINPGGSNTYYANSVKGRFTISRDNAKTTLYLHMDSLKPEDTAVYYCAQGLRVSSTKGQGTQVTVSS

SEQ ID NO: 228 - protein, 116 aa, synthetic construct
  REGION 1..116: note = nanobody sequence
QVQLQESGGGLVQAGGSLRLSCAASGFAFSTYVMTWVRQAPGKGLEWVSSINPGGSNTYYANSVKGRFTISRDNAKTTLYLHMDSLKPEDTAVYYCAQGLRVSSTKGQGTQVTVSS

SEQ ID NO: 229 - protein, 117 aa, synthetic construct
  REGION 1..117: note = nanobody sequence
QVQLQEWGGGLVQTGAALRLSCGASGRIFNIYATGWFRQAPGKEREFVARISASGDTSYADSVKGRFTISRDYSKNTVYLQMNSLNSEDTAVYYCAANQRGTGYQYWGRGTQVTVSS

SEQ ID NO: 230 - protein, 117 aa, synthetic construct
  REGION 1..117: note = nanobody sequence
QVQLQESGGGLVQTGDSLRLSCGASGRIFNIYATGWFRQAPGKEREFVARISASGDTSYADSVKGRFTISRDYSKNTVYLQMNSLNSEDTAVYYCAANQRGTGYQYWGQGTQVTVSS

SEQ ID NO: 231 - protein, 119 aa, synthetic construct
  REGION 1..119: note = nanobody sequence
QVQLQESGGGLVQAGGSLRLSCAASGRTFSSYAMGWFRQAPGKEREFLVAISWSGDITYYADSVKGRFTISRDNAKNTVYLHMNSLKPEDTAVYYCAAQPLIGQTYDYWGQGTQVTVSS

SEQ ID NO: 232 - protein, 119 aa, synthetic construct
  REGION 1..119: note = nanobody sequence
QVQLQESGGGLVQAGGSLRLSCAASGRTFSSYAMGWFRQAPGKEREFLVAISWSGDITYYADSVKGRFTISRDNAKNTVYLQMNSLKPEDTAVYYCAAQPLIGQTYDYWGQGTQVTVSS

SEQ ID NO: 233 - protein, 123 aa, synthetic construct
  REGION 1..123: note = nanobody sequence
QVQLQESGGGLVQVGDSLRLSCAASGVTFSPYAVGWFRQAPGKEREFVGFMHWYGGIFYTNSVKGRFRISRDNAKNTVYLEMNRLKPEDTAVYYCAARSGSEGSSLRSEYDYWGQGTQVTVSS

SEQ ID NO: 234 - protein, 123 aa, synthetic construct
  REGION 1..123: note = nanobody sequence
QVQLQESGGGLVQVGDSLRLSCAASGVTSSPYAVGWFRQAPGKEREFVGFMHWYGGVYYTNSVKGRFTISRDNAKNTVYLEMNRLKPEDTAVYYCAARSGSEGSHLRYEYDYWGQGTQVTVSS

SEQ ID NO: 235 - protein, 125 aa, synthetic construct
  REGION 1..125: note = nanobody sequence
QVQLQESGGGLLQAGDSLRLSCAASGRDFTTTSVGWFRQAPGKEREFVTSISPNGGTTKYADSVKGRFTISRNNAKNTVYLQMNSLKVEDTAVYYCAAGPYYAAAVTSLRAYTDWGQGTQVTVSS

SEQ ID NO: 236 - protein, 125 aa, synthetic construct
  REGION 1..125: note = nanobody sequence
QVQLQESGGGLVQAGDSLRLSCAASGRDFTTTSVGWFRQAPGKEREFVTSISPNGGTTKYADSVKGRFTISRNNAKNTVYLQMNSLKVEDTAVYYCAAGPYYAAAVTSLRAYTDWGQGTQVTVSS

SEQ ID NO: 237 - protein, 126 aa, synthetic construct
  REGION 1..126: note = nanobody sequence
QVQLQESGGGLVQAGGSLGLSCAFSGGTFTDHDMGWFRQTQEKERELVVRISWSGNTRNYADPVKGRFTISRDNAKNTVSLQMNSLKPEDTAVYYCAARHPAAYGSYSDQSTYYAWGQGTQVTVSS

SEQ ID NO: 238 - protein, 126 aa, synthetic construct
  REGION 1..126: note = nanobody sequence
QVQLQESGGGLVQAGGSLRLSCAFSGGTFTDHDMGWFRQTQEKERELVVRISWSGNTRNYADPVKGRFTISRDNAKNTVSLQMNSLKPEDTAVYYCAARHPAAYGSYSDQSTYYAWGQGTQVTVSS

SEQ ID NO: 239 - protein, 126 aa, synthetic construct
  REGION 1..126: note = nanobody sequence
QVQLQESGGGSVQAGGSLRLSCAASGRTVSGLAMGWFRQAPGKEREVVAGISWNGASTYYADSVKGRFTISRDNAENTVYLQMNSLKPEDTAVYYCAADRFSSTVVRRRPAEYDYWGQGTQVTVSS

SEQ ID NO: 240 - protein, 126 aa, synthetic construct
  REGION 1..126: note = nanobody sequence
QVQLQESGGGLVQAGGSLRLSCAASGRTVSGSAMGWFRQAPGKEREVVAGISWNGASTYYADSVKGRFTISRDNAENTVYLQMNSLKPEDTAVYYCAADRFSSTVVRRRPAEYDYWGQGTQVTVSS

SEQ ID NO: 241 - protein, 111 aa, synthetic construct
  REGION 1..111: note = nanobody sequence
QVQLQESGGGLVQPGGSLRLSCAASALTFSVRPINWYRQAPGKRREVVATITSSGSTWYVDFVKGRFTISRDNAKNMAYLQMNSLKPEDTAVYYCNDGPLWGQGTQVTVSS

SEQ ID NO: 242 - protein, 112 aa, synthetic construct
  REGION 1..112: note = nanobody sequence
QVQLQESGGGLVQPGGSLRLSCAASGFTFSRDWMTWVRQGPGKGLEWVAAIDSDGTTYYADSMKGRFTISKDNAKNTVYLQLNNLQPDDTAIYKCKMGGSGWGQGTQVTVSS

SEQ ID NO: 243 - protein, 115 aa, synthetic construct
  REGION 1..115: note = nanobody sequence
QVQLQESGGGSVQTGESLRLSCAASGFTFSSFAMSWVRQAPGKGLEWVSTIHSPSSHTWYADSVKGRFTISTDNAKNTLYLQMNNLKPEDTAVYYCTKEGRSDSKGPGTQVTVSS

SEQ ID NO: 244 - protein, 116 aa, synthetic construct
  REGION 1..116: note = nanobody sequence
QVQLQESGGGLVQPGGSLRLSCAASGFTFSTYLMSWVRQAPGKGLEWISSINDGGGSPTYAASVKGRFTISRDNAKNTLHLQMNNLKPDDTAVYYCTTIFSGQMRRGQGTQVTVSS

SEQ ID NO: 245 - protein, 117 aa, synthetic construct
  REGION 1..117: note = nanobody sequence
QVQLQESGGGSVQAGGSLRLSCAASGRTGSIYGMGWFRQAPGKERQFVAQISWIGTNTYYEDSVKGRFTISRDNAKNTVYLQMNTLKHEDSAVYYCSAMVRGQDRYWGQGTQVTVSS

SEQ ID NO: 246 - protein, 117 aa, synthetic construct
  REGION 1..117: note = nanobody sequence
QVQLQESGGGLAQPGGSLKLSCAASGFPFSYTVMSWVRQAPGKELEWVSTITHDSTTTYYATSVEGRFTISRNNGLDTLWLQMNDLKPEDTAVYYCAKGLNWRTAQLGQGTQVTVSS

SEQ ID NO: 247 - protein, 118 aa, synthetic construct
  REGION 1..118: note = nanobody sequence
QVQLQESGGGLVQPGGSLRLSCTASGFTFSRYPMTWLRQAPGKEPEWVSSVDGHASKTWYADSVKGRFTISRDNAQNTLYLQMNSLKPEDTALYYCVRGLDTGTAPQLGQGTQVTVSS

SEQ ID NO: 248 - protein, 117 aa, synthetic construct
  REGION 1..117: note = nanobody sequence
QVQLQESGGGLVQPGGSLRLSCVTSGFVFSKNLLTWARQAPGKEPEWVSTISAHNTTWYADSVTGRFTISRDNSKNTVYLQMNNLTPEDTALYYCARGGTNAIAERLGRGTQVTVSS

SEQ ID NO: 249 - protein, 119 aa, synthetic construct
  REGION 1..119: note = nanobody sequence
QVQLQESGGGLVQAGDSLRLSCAASARTFSIYTMGWFRQAPGKEREFVAAISWSGGKTYYADSVKGRFTISRDNAENTVYLQMNSLKPEDTAVYYCGADLEGGGSYYGPGQGTQVTVSS

SEQ ID NO: 250 - protein, 120 aa, synthetic construct
  REGION 1..120: note = nanobody sequence
QVQLQESGGGLVQAGGSLRLSCAASKRIFSLYNKAWFRQAPGKEREFVAAISVSGTTINYADSVKGRFTISRDNTKNTVYLQMHSLEPEDTAVYYCAAKQNYGAGNYDYWGQGTQVTVSS

SEQ ID NO: 251 - protein, 122 aa, synthetic construct
  REGION 1..122: note = nanobody sequence
QVQLQESGGGLVQPGGSLRLSCAASGFPLDYYAIGWFRQAPGKEREGVSCISSSGGSTNYAASVKGRFTISRDNAKNTVYLQMNSLKPEDTAVYYCASMSSTWCVSWGADYWGKGTQVTVSS

SEQ ID NO: 252 - protein, 123 aa, synthetic construct
  REGION 1..123: note = nanobody sequence
QVQLQESGGGLVQAGGSLRLTCAASGRTNSGDSMGWFRQAPGKEREFVAAIRWSGTSTYYTDTVKGRFTISRDNAKNTVYLQMNSLKPEDTAVYYCAATASLRSLTSRQYDYWGRGTQVTVSS

SEQ ID NO: 253 - protein, 123 aa, synthetic construct
  REGION 1..123: note = nanobody sequence
QVQLQESGGGLVQPGGSLRLSCAASGFTISSYAMSWVRQAPGKGLEWVSGINSGGGSTSYADSVKGRFTISRDNAKNTLYLQMNSLKPEDTGVYYCQKLQYGYFPGSGAYSHRGQGTQVTVSS

SEQ ID NO: 254 - protein, 123 aa, synthetic construct
  REGION 1..123: note = nanobody sequence
  VARIANT 13: note = X can be any naturally occurring amino acid
QVQLQESGGGLVXPGGSLRLSCAASGFTFSSYYMYWVRQAPGKGLEWVSLSIPGGIRTYYAESVKGRFTISRDNARNTVNLQMSSLKPEDTAVYYCAADPRLLGSTSVSYKYRGQGTQVTVSS

SEQ ID NO: 255 - protein, 124 aa, synthetic construct
  REGION 1..124: note = nanobody sequence
QVQLQESGGGLVQAGDSLRLSCVASGGTFSRYSMGWFRQAPGKEREFVGFISWTTGSANYADSVKGRFTISRDNAKNTMNLQMNSLKPEDTAVYYCAAGLRRGSWYYTPDYDYWGQGTQVTVSS

SEQ ID NO: 256 - protein, 125 aa, synthetic construct
  REGION 1..125: note = nanobody sequence
QVQLQESGGGLVQTGGSLRLSCAASGSTFSSYAMGWFRQGPGKEREFVAAISWSGGTKYYADSVKGRFTISRDNAKNTVYLQMNSLKPEDTAAYYCAADPVGGPGYDHINAYDYWGQGTQVTVSS

SEQ ID NO: 257 - protein, 126 aa, synthetic construct
  REGION 1..126: note = nanobody sequence
QVQLQESGGGLVQPGGSLRLSCSASGLTFSTYTMAWFRQAPGKEREFVAGISWSGTNPYYADSVKGRFTLSRDSAKNTVYLQLNSLKPEDTALYYCAADRRLLYTLVYKANENTYWGQGTQVTVSS

SEQ ID NO: 258 - protein, 126 aa, synthetic construct
  REGION 1..126: note = nanobody sequence
QVQLQESGGGLVQAGGSLRLSCAASGFTFDDYAITWFRQAPGKEREGVSCISSSDGSTYYADSVKGRFTISSDNAKNTVYLQMNSLKPEDTAVYYCAGAEGIYYSGSCSYIESDYWGQGTQVTVSS

SEQ ID NO: 259 - protein, 127 aa, synthetic construct
  REGION 1..127: note = nanobody sequence
  VARIANT 39: note = X can be any naturally occurring amino acid
QVQLQESGGGLVQAGGSLRLSCAASGRTFSTIGMGWFRXAPGKEREFVAHISWSAGSTYYADSVKGRFTISRDNAKITVYLHMNSLEPEDTAVYYCVADVRNNGRESGLGSSSARYWGQGTQVTVSS

SEQ ID NO: 260 - protein, 19 aa, synthetic construct
  REGION 1..19: note = amino terminal HA epitope and polyhistidine tag
AAAYPYDVPDYGSHHHHHH

SEQ ID NO: 261 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0000795 A1
(43) Pub. Date: Jan. 1, 2026

(54) HER3 NANOBODIES

(71) Applicant: ACTINIUM PHARMACEUTICALS, INC., New York, NY (US)

(72) Inventors: GHOLAMREZA HASSANZADEH GHASSABEH, Sint-Genesius-Rode (BE); Steve SCHOONOOGHE, Holsbeek (BE); Helen KOTANIDES, Norwalk, CT (US)

(73) Assignee: ACTINIUM PHARMACEUTICALS, INC., New York, NY (US)

(21) Appl. No.: 18/846,463

(22) PCT Filed: Mar. 24, 2023
(86) PCT No.: PCT/US2023/064968

§ 371 Date: Sep. 12, 2024

Related U.S. Application Data
(60) Provisional application No. 63/323,471, filed on Mar. 24, 2022.

(51) Int. Cl. A61K 51/10; C07K 16/32

(52) U.S. Cl. CPC A61K 51/1093; A61K 51/1045; C07K 16/32; C07K 2317/52; C07K 2317/565; C07K 2317/569; C07K 2319/00

(57) ABSTRACT

Provided are human HER3 binding nanobodies and pharmaceutical compositions including the nanobodies.

### CROSS-REFERENCE TO RELATED APPLICATIONS

This application is the § 371 U.S. national stage of International application serial no. PCT/US2023/064968 filed Mar. 24, 2023, which claims priority to U.S. provisional application Ser. No. 63/323,471 filed Mar. 24, 2022, each of which is hereby incorporated by reference in its entirety.

### SEQUENCE LISTING

The instant application contains a Sequence Listing which has been submitted electronically in XML format and is hereby incorporated by reference in its entirety. Said XML copy, created on Jul. 29, 2025 is named ATNM-017PCT_SL_ST26.xml and is 338,368 bytes in size.

### FIELD OF THE INVENTION

The presently claimed invention relates to the field of nanobody-based therapeutics.

### BACKGROUND

HER3 is a protein over expressed on a number of cancers including breast cancer, such as tamoxifen-resistant breast cancer and triple negative breast cancers (TNBC), prostate cancer, metastatic castration resistant prostate cancer (mCRPC), renal cell carcinoma, hepatocellular carcinoma, colorectal cancer, ovarian cancer, head and neck carcinoma, lung cancer, non-small cell carcinoma, urothelial cancer, glioma, endometrial cancer, urothelial cancer, melanoma, thyroid cancer, cervical cancer, pancreatic cancer, gastric cancer, and testicular cancer.

What is needed and provided by the various aspects of the present invention are new human-HER3 targeting agents.

### SUMMARY OF THE INVENTION

One aspect of the invention provides an anti-huHER3 nanobody or a fusion protein including an anti-huHER3 nanobody amino acid sequence, the nanobody or fusion protein including:
(i) a nanobody amino acid sequence including the CDRs (CDR1, CDR2 and CDR3) of any of the nanobodies disclosed herein;
(ii) a nanobody amino acid sequence including the framework regions and the CDRs of any of the nanobodies disclosed herein; or
(iii) a nanobody amino acid sequence including the full nanobody amino acid sequence of any of the nanobody sequences disclosed herein.

Additional features, advantages, and aspects of the invention may be set forth or apparent from consideration of the following detailed description, drawings if any, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1A identifies the full amino acid sequence, the CDR3 group, and the CDR amino acid sequences for each of twenty-four (24) human HER3 binding nanobody clones.

FIG. 1B identifies the full amino acid sequence, the CDR3 group, and the CDR amino acid sequences for each of twenty-four (24) human HER3 binding nanobody clones.

FIG. 1C identifies the full amino acid sequence, the CDR3 group, and the CDR amino acid sequences for each of twenty-four (24) human HER3 binding nanobody clones.

FIG. 1D identifies the full amino acid sequence, the CDR3 group, and the CDR amino acid sequences for each of twenty-four (24) human HER3 binding nanobody clones.

FIG. 1E identifies the full amino acid sequence, the CDR3 group, and the CDR amino acid sequences for each of six (6) human HER3 binding nanobody clones.

### DETAILED DESCRIPTION

A nanobody (Nb) or VHH domain antibody is the variable region of a camelid heavy chain-only antibody. The present invention provides nanobodies and nanobody fusion proteins that specifically bind human HER3 (huHER3) and related compositions and methods of use thereof.

One aspect of the invention provides an anti-huHER3 nanobody or a fusion protein including an anti-huHER3 nanobody amino acid sequence, the nanobody or fusion protein including:
(i) a nanobody amino acid sequence including the CDRs (CDR1, CDR2 and CDR3) of any of the nanobodies disclosed herein;
(ii) a nanobody amino acid sequence including the framework regions and the CDRs of any of the nanobodies disclosed herein; or
(iii) a nanobody amino acid sequence including the full nanobody amino acid sequence of any of the nanobody sequences disclosed herein.

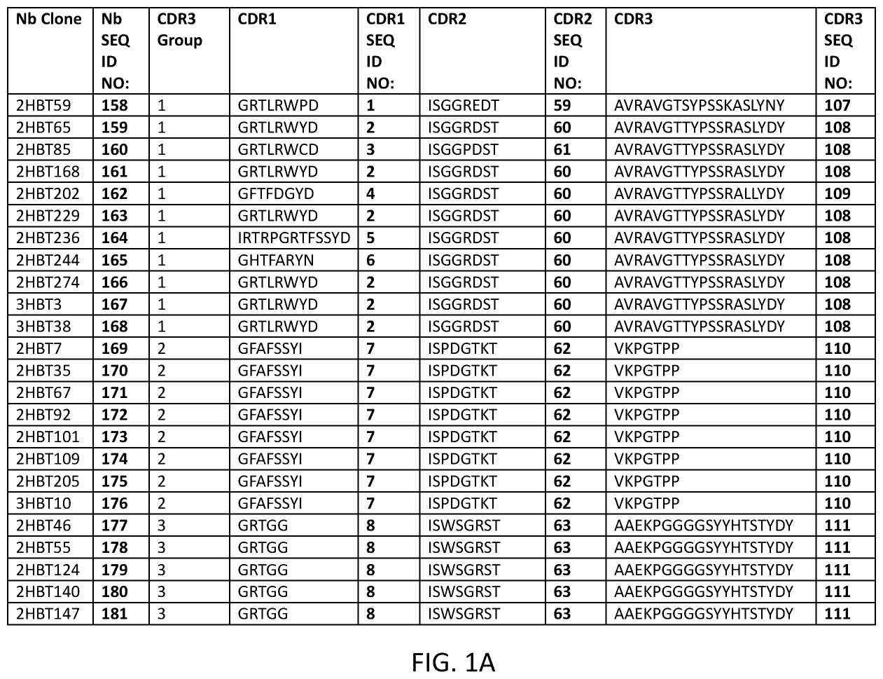

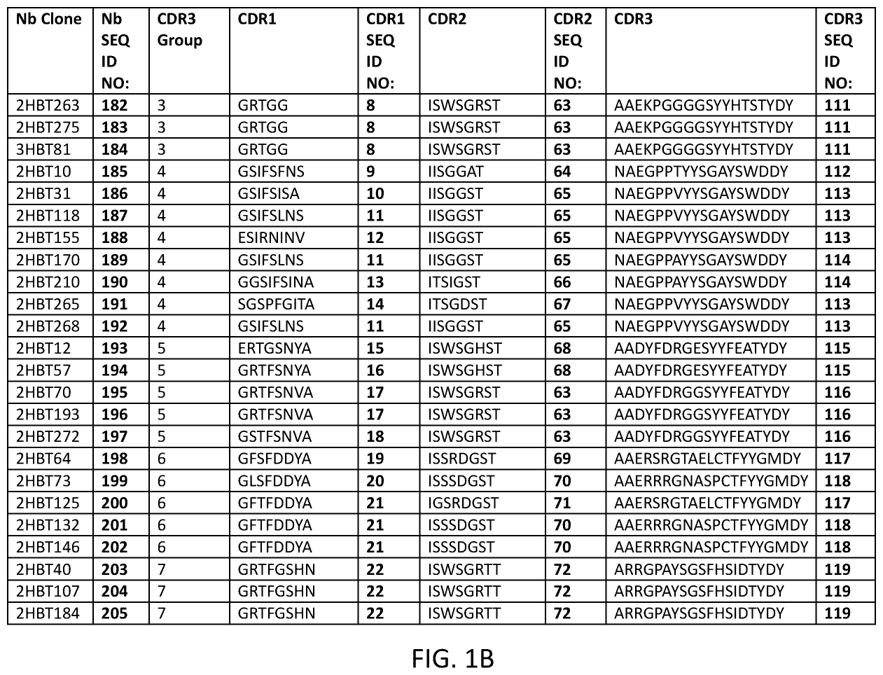

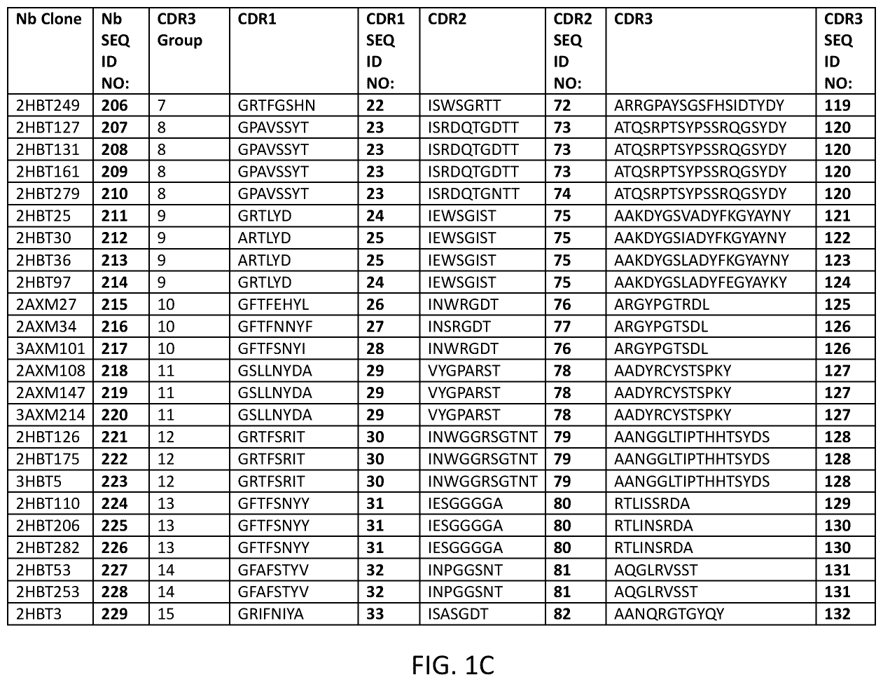

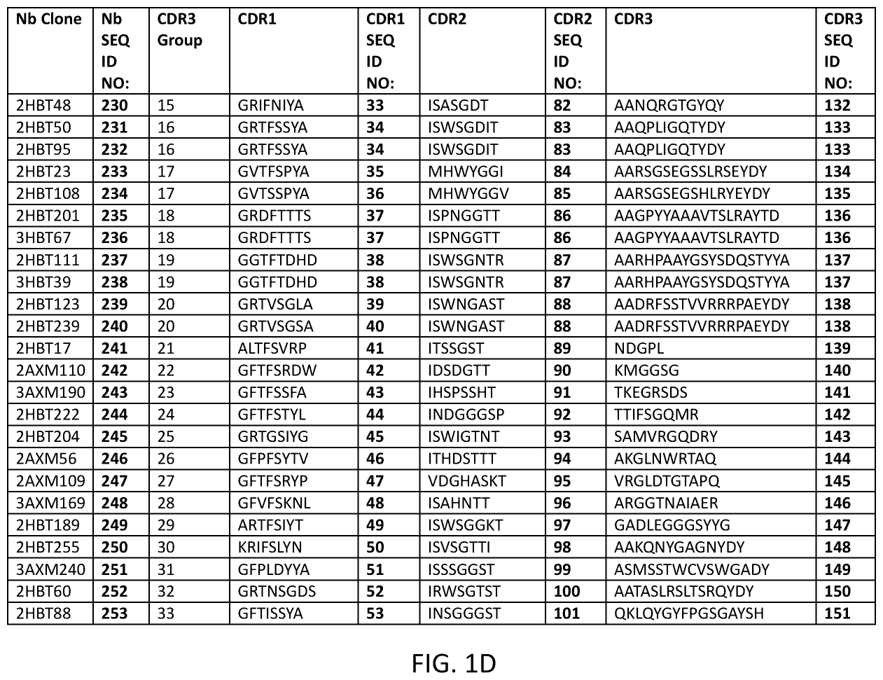

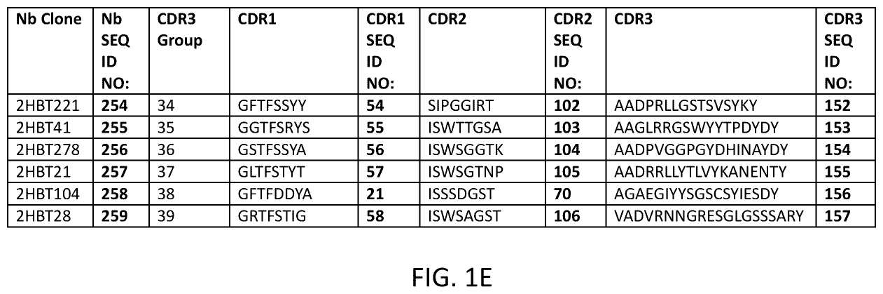

FIG. 1A identifies the full amino acid sequence (SEQ ID NOS:158-181, respectively), the CDR3 group, and the CDR amino acid sequences for each of the human HER3 binding nanobody clones designated 2HBT59, 2HBT65, 2HBT85, 2HBT168, 2HBT202, 2HBT229, 2HBT236, 2HBT244, 2HBT274, 3HBT3, 3HBT38, 2HBT7, 2HBT35, 2HBT67, 2HBT92, 2HBT101, 2HBT109, 2HBT205, 3HBT10, 2HBT46, 2HBT55, 2HBT124, 2HBT140, and 2HBT147.

FIG. 1B identifies the full amino acid sequence (SEQ ID NOS:182-205, respectively), the CDR3 group, and the CDR amino acid sequences for each of the human HER3 binding nanobody clones designated 2HBT263, 2HBT275, 3HBT81, 2HBT10, 2HBT31, 2HBT118, 2HBT155, 2HBT170, 2HBT210, 2HBT265, 2HBT268, 2HBT12, 2HBT57, 2HBT70, 2HBT193, 2HBT272, 2HBT64, 2HBT73, 2HBT125, 2HBT132, 2HBT146, 2HBT40, 2HBT107, and 2HBT184.

FIG. 1C identifies the full amino acid sequence (SEQ ID NOS:206-229, respectively), the CDR3 group, and the CDR amino acid sequences for each of the human HER3 binding nanobody clones designated 2HBT249, 2HBT127, 2HBT131, 2HBT161, 2HBT279, 2HBT25, 2HBT30, 2HBT36, 2HBT97, 2AXM27, 2AXM34, 3AXM101, 2AXM108, 2AXM147, 3AXM214, 2HBT126, 2HBT175, 3HBT5, 2HBT110, 2HBT206, 2HBT282, 2HBT53, 2HBT253, and 2HBT3.

FIG. 1D identifies the full amino acid sequence (SEQ ID NOS:230-253, respectively), the CDR3 group, and the CDR amino acid sequences for each of the human HER3 binding nanobody clones designated 2HBT48, 2HBT50, 2HBT95, 2HBT23, 2HBT108, 2HBT201, 3HBT67, 2HBT111, 3HBT39, 2HBT123, 2HBT239, 2HBT17, 2AXM110, 3AXM190, 2HBT222, 2HBT204, 2AXM56, 2AXM109, 3AXM169, 2HBT189, 2HBT255, 3AXM240, 2HBT60, and 2HBT88.

FIG. 1E identifies the full amino acid sequence (SEQ ID NOS:254-259, respectively), the CDR3 group, and the CDR amino acid sequences for each of the human HER3 binding nanobody clones designated 2HBT221, 2HBT41, 2HBT278, 2HBT21, 2HBT104, and 2HBT28.

The CDR sequences of the nanobody clones are delineated according to the IMGT numbering convention. The CDRs are surrounded by VHH domain framework regions (FRs) in the following manner: FR1 is the amino acid sequence preceding (N-terminal to) CDR1, FR2 is the amino acid sequence between CDR1 and CDR2, FR3 is the amino acid sequence between CDR2 and CDR3, and FR4 is the amino acid sequence following (C-terminal to) CDR3 to the end of the nanobody (VHH domain) sequence.

Any of the nanobodies disclosed herein may further include an affinity tag such as an epitope tag and/or a metal-binding tag. For example, any of the nanobodies disclosed herein may further include an amino terminal combination hemagglutinin (HA) epitope and polyhistidine tag having the sequence AAAYPYDVPDYGSHHHHHH (SEQ ID NO: 260).

The invention also provides fusion proteins that include any of the HER3-binding nanobodies disclosed herein and an N-terminal, camelid or non-camelid immunoglobulin Fc region, such as a human immunoglobulin Fc region such as the human IgG1 Fc region set forth in SEQ ID NO:261.

Another aspect of the invention provides a protein that includes one or more of the human HER3 binding nanobody amino acid sequences set forth in SEQ ID NOS:158-259. Such a protein may, for example, consist of one nanobody amino acid sequence alone, include multiple nanobody sequences that are the same or different, or include the one or more of the nanobody sequences and an affinity tag, such as an epitope tag and/or metal-binding tag, or include an Fc constant region, such as a human Fc constant region.

A related aspect of the invention provides a protein, such as a nanobody or a fusion protein including a nanobody amino acid sequence, that includes a nanobody (VHH) amino acid sequence including the CDR combination (of CDR1, CDR2, and CDR3) found in any one of the anti-huHER3 nanobody sequences set forth in SEQ ID NOS:158-259 as shown in FIGS. 1A-1E, and in Table 1 below (indicating exemplary human HER3 binding nanobody sequences and, to the right of each in the table, its respective CDR1, CDR2, and CDR3 amino acid sequences).

TABLE 1

| Nb | CDR1 | CDR2 | CDR3 |
|---|---|---|---|
| SEQ | SEQ | SEQ | SEQ |
| ID NO: | ID NO: | ID NO: | ID NO: |
| 158 | 1 | 59 | 107 |
| 159 | 2 | 60 | 108 |
| 160 | 3 | 61 | 108 |
| 162 | 4 | 60 | 109 |
| 164 | 5 | 60 | 108 |
| 165 | 6 | 60 | 108 |
| 169 | 7 | 62 | 110 |
| 170 | 7 | 62 | 110 |
| 177 | 8 | 63 | 111 |
| 185 | 9 | 64 | 112 |
| 186 | 10 | 65 | 113 |
| 187 | 11 | 65 | 113 |
| 188 | 12 | 65 | 113 |
| 189 | 11 | 65 | 114 |
| 190 | 13 | 66 | 114 |
| 191 | 14 | 67 | 113 |
| 193 | 15 | 68 | 115 |
| 194 | 16 | 68 | 115 |
| 195 | 17 | 63 | 116 |
| 197 | 18 | 63 | 116 |
| 198 | 19 | 69 | 117 |
| 199 | 20 | 70 | 118 |
| 200 | 21 | 71 | 117 |
| 200 | 21 | 71 | 117 |
| 201 | 21 | 70 | 118 |
| 203 | 22 | 72 | 119 |
| 206 | 22 | 72 | 119 |
| 207 | 23 | 73 | 120 |
| 210 | 23 | 74 | 120 |
| 211 | 24 | 75 | 121 |
| 212 | 25 | 75 | 122 |
| 213 | 25 | 75 | 123 |
| 214 | 24 | 75 | 124 |
| 215 | 26 | 76 | 125 |
| 216 | 27 | 77 | 126 |
| 217 | 28 | 76 | 126 |
| 218 | 29 | 78 | 127 |
| 221 | 30 | 79 | 128 |
| 224 | 31 | 80 | 129 |
| 225 | 31 | 80 | 130 |
| 227 | 32 | 81 | 131 |
| 229 | 33 | 82 | 132 |
| 231 | 34 | 83 | 133 |
| 233 | 35 | 84 | 134 |
| 234 | 36 | 85 | 135 |
| 237 | 38 | 87 | 137 |
| 239 | 39 | 88 | 138 |
| 240 | 40 | 88 | 138 |
| 241 | 41 | 89 | 139 |
| 242 | 42 | 90 | 140 |
| 243 | 43 | 91 | 141 |
| 244 | 44 | 92 | 142 |
| 245 | 45 | 93 | 143 |
| 246 | 46 | 94 | 144 |
| 247 | 47 | 95 | 145 |
| 248 | 48 | 96 | 146 |
| 249 | 49 | 97 | 147 |
| 250 | 50 | 98 | 148 |
| 251 | 51 | 99 | 149 |
| 252 | 52 | 100 | 150 |
| 253 | 53 | 101 | 151 |
| 254 | 54 | 102 | 152 |
| 255 | 55 | 103 | 153 |
| 256 | 56 | 104 | 154 |
| 257 | 57 | 105 | 155 |
| 258 | 21 | 70 | 156 |
| 259 | 58 | 106 | 157 |

The nanobodies and fusion proteins including such nanobodies as disclosed herein may, for example, be linked directly or indirectly via a chemically conjugated chelator, to a radionuclide, for example, to target cytotoxic radiation to BER3-expressing cells in mammalian subject such as a human patient, or to non-cytotoxically image BER3-expression in a mammalian subject such as a human patient. For example, the nanobody or fusion protein may be directly labeled with ¹³¹I according to the methods disclosed in U.S. Pat. No. 10,420,851 or the antibody may be chemically conjugated to a chelator, such as p-SCN-DOTA and labeled with a radionuclide ²²⁵Ac, according to the procedures described in U.S. Pat. No. 9,603,954.

The nanobodies or fusion proteins including a nanobody may, for example, also be linked to one or more cytotoxic drugs to target and deplete HER3-expressing cells in a mammalian subject such as a human patient. Thus, one aspect of the invention provides an antibody-drug-conjugate (ADC) that includes a nanobody or nanobody fusion protein as disclosed herein as a component.

The radionuclide may, for example, selected from ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ⁸⁹Zr, ⁹⁷Ru, ¹⁰⁵Ph, ¹⁰⁹Pd, ¹¹¹In, ¹⁰⁹Pd, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, and, ²²⁷Th.

The chelator group in the various aspects of the invention may, for example, include: 1,4,7,10-tetraazacyclododecane-1,4,7-triacetic acid (DO3A) or a derivative thereof, 1,4,7-triazacyclononane-1,4-diacetic acid (NODA) or a derivative thereof, 1,4,7-triazacyclononane-1,4,7-triacetic acid (NOTA) or a derivative thereof; 1,4,7,10-tetraazacyclododecane-1,4,7,10-tetraacetic acid (DOTA) or a derivative thereof, 1,4,7-triazacyclononane, 1-glutaric acid-4,7-diacetic acid (NODAGA) or a derivative thereof; 1,4,7,10-tetraazacyclodecane, 1-glutaric acid-4,7,10-triacetic acid (DOTAGA) or a derivative thereof; 1,4,8,11-tetraazacyclotetradecane-1,4,8,11-tetraacetic acid (TETA) or a derivative thereof, 1,4,8,11-tetraazabicyclo[6.6.2]hexadecane-4,11-diacetic acid (CB-TE2A) or a derivative thereof; diethylene triamine pentaacetic acid (DTPA), its diester, or a derivative thereof; 2-cyclohexyl diethylene triamine pentaacetic acid (CHX-A″-DTPA) or a derivative thereof, deforoxamine (DFO) or a derivative thereof, 1,2-[[6-carboxypyridin-2-yl]methylamino]ethane (H₂dedpa) or a derivative thereof, DADA or a derivative thereof; 1,4,7,10-Tetraazacyclododecane-1,4,7,10-tetra(methylene phosphonic acid) (DOTP) or a derivative thereof; 4-amino-6-[[16-[(6-carboxypyridin-2-yl)methyl]-1,4,10,13-tetraoxa-7,16-diazacyclooctadec-7-yl]methyl]pyridine-2-carboxylic acid (MACROPA-NH₂) or a derivative thereof, MACROPA or a derivative thereof, 1,4,7,10-tetrakis(carbamoylmethyl)-1,4,7,10-tetraazacyclododecane (TCMC) or a derivative thereof; {4-[2-(bis-carboxymethylamino)-ethyl]-7-carboxymethyl-[1,4,7]triazonan-1-yl}-acetic acid (NETA) or a derivative thereof, Diamsar or a derivative thereof; 1,4,7-triazacyclononane-1,4,7-tris[methyl(2-carboxyethyl)phosphinic acid (TRAP, PRP9, TRAP-Pr) or a derivative thereof; N,N′-bis(6-carboxy-2-pyridylmethyl)ethylenediamine-N,N′-diacetic acid (H4octapa) or a derivative thereof; N,N′-[1-benzyl-1,2,3-triazole-4-yl]methyl-N,N′-[6-(carboxy)pyridin-2-yl]-1,2-diaminoethane (H2azapa) or a derivative thereof; N,N″-[[6-(carboxy)pyridin-2-yl]methyl]diethylenetriamine-N,N′,N″-triacetic acid (H5decapa) or a derivative thereof, N,N′-bis(2-hydroxy-5-sulfobenzyl)ethylenediamine-N,N′-diacetic acid (SHBED) or a derivative thereof; N,N′-bis(2-hydroxybenzyl)ethylenediamine-N,N′-diacetic acid (HBED) or a derivative thereof; 3,6,9,15-tetraazabicyclo[9.3.1]pentadeca-1(15),11,13-triene-3,6,9,-triacetic acid (PCTA) or a derivative thereof; desferrioxamine B (DFO) or a derivative thereof; N,N′-(methylenephosphonate)-N,N′-[6-(methoxycarbonyl)pyridin-2-yl]methyl-1,2-diaminoethane (H6phospa) or a derivative thereof; 1,4,7,10,13,16-hexaazacyclohexadecane-N,N′,N″,N′″,N″″,N′″″-hexaacetic acid (HEHA) or a derivative thereof, 1,4,7,10,13-pentaazacyclopentadecane-N,N′,N″,N′″,N″″-pentaacetic acid (PEPA) or a derivative thereof, or 3,4,3-LI(1,2-HOPO) or a derivative thereof.

The words “comprising” and forms of the word “comprising” as well as the word “including” and forms of the word “including,” as used in this description and in the claims, do not limit the inclusion of elements beyond what is referred to. Additionally, although throughout the present disclosure various aspects or elements thereof are described in terms of “including” or “comprising,” corresponding aspects or elements thereof described in terms of “consisting essentially of” or “consisting of” are similarly disclosed. For example, while certain aspects of the invention have been described in terms of a method “including” or “comprising” administering a radiolabeled targeting agent, corresponding methods instead reciting “consisting essentially of” or “consisting of” administering the radiolabeled target are also within the scope of said aspects and disclosed by this disclosure.

In addition, compositions including a radiolabeled anti-HER3 nanobody or anti-HER3 nanobody fusion protein may include one or more pharmaceutically acceptable carriers or pharmaceutically acceptable excipients. Such carriers are well known to those skilled in the art. For example, injectable drug delivery systems include solutions, suspensions, gels, microspheres and polymeric injectables, and can include excipients such as solubility-altering agents (e.g., ethanol, propylene glycol and sucrose) and polymers (e.g., polycaprylactones and PLGA's). An exemplary formulation may be as substantially described in U.S. Pat. No. 10,420,851 or International Pub. No. WO 2017/155937, incorporated by reference herein. For example, according to certain aspects, the formulation may include 0.5% to 5.0% (w/v) of an excipient selected from the group consisting of ascorbic acid, polyvinylpyrrolidone (PVP), human serum albumin (HSA), a water-soluble salt of HSA, and mixtures thereof. Certain formulations may include 0.5-5% ascorbic acid; 0.5-4% polyvinylpyrrolidone (PVP); and the monoclonal antibody in 50 mM PBS buffer, pH 7.

The anti-huHER3 nanobodies and nanobody fusion proteins disclosed herein may, for example, be labeled with a radionuclide, such as ¹³¹I, ¹⁷⁷Lu, or ²²⁵Ac, or conjugated to a cytotoxic drug, for use in the treatment of a HER3-expressing cancer in a mammal, such as human patient, such as a breast cancer, such as tamoxifen-resistant breast cancer and triple negative breast cancer (TNBC), prostate cancer, metastatic castration resistant prostate cancer (mCRPC), renal cell carcinoma, hepatocellular carcinoma, colorectal cancer, ovarian cancer, head and neck carcinoma, lung cancer, non-small cell lung carcinoma, urothelial cancer, glioma, endometrial cancer, urothelial cancer, melanoma, thyroid cancer, cervical cancer, pancreatic cancer, gastric cancer, or testicular cancer.

### Example 1: Production of a Radiolabeled Anti-huHER3 Nanobody

Conjugation to a chelator: A vial of lyophilized p-SCN-Bn-DOTA is reconstituted with metal-free water to a concentration of 10 mg/ml. To the actinium reaction vial, 0.02 ml of ascorbic acid solution (150 mg/ml) and 0.05 ml of reconstituted p-SCN-Bn-DOTA are added and the pH adjusted to between 5 and 5.5 with 2M tetramethylammonium acetate (TMAA). The mixture is then heated at 55±4° C. for 30 minutes.

To determine the labeling efficiency of the ²²⁵Ac-p-SCN-Bn-DOTA, an aliquot of the reaction mixture is removed and applied to a 1 ml column of Sephadex C25 cation exchange resin. The product is eluted in 2-4 ml fractions with a 0.9% saline solution. The fraction of ²²⁵Ac activity that elutes is ²²⁵Ac-p-SCN-Bn-DOTA and the fraction that is retained on the column is un-chelated, unreactive ²²⁵Ac. Typically, the labeling efficiency is greater than 95%.

To the reaction mixture, 0.22 ml of previously prepared anti-HER3 Nb in DTPA (1 mg) and 0.02 ml of ascorbic acid are added. The DTPA is added to bind any trace amounts of metals that may compete with the labeling of the antibody. The ascorbic acid is added as a radio-protectant. The pH is adjusted with carbonate buffer to pH 8.5-9. The mixture is heated at 37±3° C. for 30 minutes.

The final product may be purified by size exclusion chromatography resin and eluted with 2 ml of 1% HSA.

Radiolabeling: The antibody may be conjugated to a linker, such as any of the linkers described in the above indicated patent applications. An exemplary linker includes at least dodecane tetraacetic acid (DOTA), wherein a goal of the conjugation reaction is to achieve a DOTA-antibody ratio of 3:1 to 5:1. Chelation with the radionuclide, such as ¹⁷⁷Lu, ⁹⁰Y, or ²²⁵Ac may then be performed and efficiency and purity of the resulting radiolabeled antibody, such as an anti-HER3 nanobody, may be determined by HPLC and iTLC.

An exemplary labeling reaction for ²²⁵Ac is as follows: A reaction including 15 μl 0.15M NH₄OAc buffer, pH=6.5 and 2 μL (10 μg) DOTA-anti-HER3 (5 mg/ml) may be mixed in an Eppendorf reaction tube, and 4 μL ²²⁵Ac (10 μCi) in 0.05 M HCl subsequently added. The contents of the tube may be mixed with a pipette tip and the reaction mixture incubated at 37° C. for 90 min with shaking at 100 rpm. At the end of the incubation period, 3 μL of a 1 mM DTPA solution may be added to the reaction mixture and incubated at room temperature for 20 min to bind the unreacted ²²⁵Ac into the ²²⁵Ac-DTPA complex. Instant thin layer chromatography with 10 cm silica gel strip and 10 mM EDTA/normal saline mobile phase may be used to determine the radiochemical purity of ²²⁵Ac-DOTA-anti-HER3 Nb through separating 225Ac-labeled anti-HER3 (²²⁵Ac-DOTA-anti-HER3 Nb) from free ²²⁵Ac (²²⁵Ac-DTPA). In this system, the radiolabeled antibody stays at the point of application and ²²⁵Ac-DTPA moves with the solvent front. The strips may be cut in halves and counted in the gamma counter equipped with the multichannel analyzer using channels 72-110 for ²²⁵Ac to exclude its daughters.

Purification: Radiolabeled nanobody may be purified using Thermo Scientific Pierce protein concentrators PES, 3K MWCO volume 0.5 mL and 2-6 mL. An exemplary radiolabeled targeting agent, such as ²²⁵Ac-DOTA-nanobody Fc fusion protein, may be purified either on PD10 columns pre-blocked with 1% HSA or on Vivaspin centrifugal concentrators with a 50 kDa MW cut-off with 2×1.5 mL washes, 3 min per spin. HPLC analyses of the ²²⁵Ac-DOTA-antibody after purification may be conducted using a Waters HPLC system equipped with flow-through Waters UV and Bioscan Radiation detectors, using a TSK3000SW XL column eluted with PBS at pH=7.4 and a flow rate of 1 ml/min. Appropriate molecular weight cutoff filters are readily selectable and available for the purification of subject radiolabeled proteins of different molecular weights.

While various specific embodiments have been illustrated and described herein, it will be appreciated that various changes can be made without departing from the spirit and scope of the invention(s). Moreover, features described in connection with one aspect of the invention may be used in conjunction with other aspects of the invention, even if not explicitly exemplified in combination within.

1. (canceled)

2. A protein comprising a human HER3 binding nanobody amino acid sequence comprising a human HER3 binding nanobody amino acid sequence comprising the CDR1, CDR2, and CDR3 sequences:
(i) SEQ ID NO:2, SEQ ID NO: 60, and SEQ ID NO:108, respectively;
(ii) SEQ ID NO:25, SEQ ID NO: 75, and SEQ ID NO:122, respectively;
(iii) SEQ ID NO:32, SEQ ID NO: 81, and SEQ ID NO:131, respectively;
(iv) SEQ ID NO:33, SEQ ID NO: 82, and SEQ ID NO:132, respectively; or
(v) SEQ ID NO:56, SEQ ID NO: 104, and SEQ ID NO:154, respectively.

3. (canceled)

4. The protein of claim 2, comprising one or more of the human HER3 binding nanobody amino acid sequences set forth in SEQ ID NOS:159, 212, 228, 230 or 256.

5. The protein of claim 2, consisting essentially of a single VHH domain.

6. The protein of claim 2, wherein the protein is a nanobody Fc fusion protein,

7. A pharmaceutical composition comprising the protein of claim 2, and at least pharmaceutically acceptable excipient.

8. A radiopharmaceutical composition comprising the protein of claim 2 linked to a radionuclide.

9. The radiopharmaceutical composition of claim 8, further comprising at least one pharmaceutically acceptable excipient.

10. The radiopharmaceutical composition of claim 9, wherein the radionuclide is an alpha particle emitter.

11. The radiopharmaceutical composition of claim 9, wherein the radionuclide is a beta particle emitter.

12. The radiopharmaceutical of claim 9, wherein the radionuclide comprises ¹³¹I.

13. The radiopharmaceutical of claim 9, wherein the radionuclide comprises ²²⁵Ac, ¹¹⁷Lu or ⁹⁰Y.

14. A composition comprising the protein of claim 2, chemically conjugated to a chelator.

15. The composition of claim 14, wherein the chelator comprises DOTA or a DOTA derivative.

16. The composition of claim 14, further comprising a radionuclide chelated by the chelator.

17. The composition of claim 15, further comprising a radionuclide chelated by the chelator.
